(19) United States
(12) Patent Application Publication
(10) Pub. No.: US 2003/0096957 A1
(43) Pub. Date: May 22, 2003

(54) AFX (zeta) transcription factor splice form

(76) Inventors: Benjamin Robert Bowen, Summit, NJ (US); James Patrick Whelan, Madison, CT (US); Zhenyu Yang, Menlo Park, CA (US)

Correspondence Address: THOMAS HOXIE, NOVARTIS CORPORATION, PATENT AND TRADEMARK DEPT, 564 MORRIS AVENUE, SUMMIT, NJ, 079011027

(21) Appl. No.: 10/186,839

(22) Filed: Jul. 1, 2002

Related U.S. Application Data
(60) Provisional application No. 60/302,134, filed on Jun. 29, 2001.

(51) Int. Cl. C07K 14/475; C07K 16/22; C12P 21/02; C12N 5/06

(52) U.S. Cl. 530/350; 530/388.25; 435/69.1; 435/325; 435/320.1; 536/23.2; 435/70.21

(57) ABSTRACT

The subject invention is related to human AFXζ genes and gene products and methods of identifying substances which agonize or antagonize the AFXζ gene or the AFXζ gene product or AFXζ regulated genes. Another aspect of the present invention concerns pharmaceutical compositions comprising such substances for the treatment of diseases related to AFXζ.

### BACKGROUND

An important role of insulin in glucose homeostasis is regulating the transcription of genes critical in glucose metabolism (1). For example, insulin inhibits the expression of genes such as phosphoenolpyruvate carboxykinase (PEPCK) (2,3), insulin-like growth factor binding protein-1 (IGFBP-1) (4,5), and the glucose-6-phosphatase catalytic subunit (G6Pase) (6). Insulin represses gene transcription through cis-acting elements known as insulin responsive sequences (IRSs) in the regulatory region of the target genes. (reviewed in (7)). The PEPCK promoter contains one IRS (8), while in the IGFBP-1 promoter two copies of IRS are arranged as an inverted palindrome (9,10). Three tandem copies of IRSs are present in the G6Pase promoter (6,1 1).

Proteins involved in the insulin signaling pathway include the insulin receptor (IR), phosphoinositol 3-kinase (PI3K), protein kinase B (PKB), and downstream transcription factors. Genetic studies suggest that an insulin-like signaling pathway exists in Caenorhabditis elegans. In the nematode, the pathway is composed of DAF-2, AGE-1, and AKT1/AKT2, which are considered the orthologs of mammalian IR (12), P13K (13), and PKB (14), respectively. Together, the products of these genes can negatively regulate the activity of DAF-16 (15), a forkhead transcription factor that binds IRS (16). Because the major target of DAF-2/AGE-1 signaling in C. elegans is DAF-16 (14,17), the orthologs of DAF-16 may represent distal effectors of insulin signaling in mammalian cells (12,15,18). The DAF-16 forkhead domain is most similar to those of human FKHR (67% identical) and AFX proteins (64% identical) (15,16). Therefore, human forkhead factors FKHR and/or AFX may be downstream targets of the insulin-activated PI3K-PKB signaling pathway and also may be responsible for mediating insulin regulation of gene expression (12,14-16).

AFX was originally identified on chromosome X as an oncogenic fusion protein in acute lymphoblastic leukemia (ALL) (19,20) and is involved in the regulation of the cell-cycle (21,22), apoptosis (23), and tumorigenesis (24). The gene for AFX consists of two exons and is reported to encode a protein of 501 amino acids (25). It is expressed ubiquitously with high levels in placenta and skeletal muscle (19). AFX binds the IRS element from IGFBP-1 and induces a pronounced increase in the activity of a reporter gene under the control of the IGFBP-1 promoter. This transcriptional activation requires an intact IRS, and insulin treatment suppresses the activation through the PI3K-PKB signaling pathway (26). Indeed, AFX contains three putative PKB phosphorylation sites and can be phosphorylated by PKB both in vitro and in vivo (23,26). Phosphorylation by PKB alters the nuclear import of AFX, shifting its localization from the nucleus to the cytoplasm and thereby inhibiting AFX transcriptional activity (27).

The PI3K-PKB pathway is an important but not unique route by which the activity of forkhead transcription factors are regulated (28,29). A PI3K inhibitor or a dominant-negative PKB mutant decreases but does not abolish insulin-induced phosphorylation of AFX (26), suggesting the existence of an alternate signaling pathway. A candidate for this alternative pathway is the mammalian AMP-activated protein kinase (AMPK) cascade. AMPK influences many metabolic processes that become dysregulated in the diabetic state (30). However, the mechanism by which AMPK modulates transcriptional activity is unknown due to the fact that the transcription factor(s) involved remains unidentified.

AFXζ is an isoform of AFX and reporter gene assays demonstrated that AFXζ is a potent transcription activator with properties distinct from those of the previously described isoform, AFXα, and that AFXζ is regulated by the insulin signaling pathway and by an agent known to affect AMPK activity.

Accordingly, there exists a need for methods for identifying substances which regulate the biological function of the AFXζ gene products, so that they may be administered to patients in need of such treatment.

There also exists a need for pharmaceuticals comprising the factors which regulate AFXζ gene product such that they may be administered to a patient in need thereof for the treatment and/or prevention of diseases mediated by the AFXζ gene product.

The gene and gene product of AFXζ is referred to herein as “AFXζ”, “AFXζ cDNA”, “AFXζ mRNA”, “human AFXζ”, “AFXζ protein” and “AFXζ polypeptide”.

### SUMMARY

Toward these ends, and others, an aspect of the present invention provides polynucleotides that encode AFXζ protein. In a preferred embodiment of this aspect of the invention the polynucleotide comprises the region encoding human AFXζ in the sequence set out in SEQ ID NO:1. In accordance with this aspect of the invention there are provided isolated nucleic acid molecules encoding human AFXζ including mRNAs, cDNAs and, in further embodiments of this aspect of the invention, biologically, diagnostically, clinically or therapeutically useful variants, analogs or derivatives thereof, or fragments thereof, including fragments of the variants, analogs and derivatives.

It also is an object of the invention to provide human AFXζ polypeptides, particularly human AFXζ polypeptides. In a preferred embodiment the polypeptide comprises the sequence shown in SEQ ID NO:2. In accordance with this aspect of the invention there are provided novel polypeptides of human origin as well as biologically, diagnostically or therapeutically useful fragments, variants and derivatives thereof, variants and derivatives of the fragments, and analogs of the foregoing.

In another aspect of the invention provides assay techniques for identifying human AFXζ agonists and antagonists and methods for identifying such agonists and antagonists. Agonists augment AFXζ action, wherein antagonists reduce or prevent the effect of human AFXζ polypeptide. Agonists augment AFXζ biological action by binding to the gene product directly or by acting on enzymes and other molecules which regulate AFXζ action. Among preferred antagonists are those which mimic human AFXζ so as to bind to human AFXζ binding molecules but do not elicit a human AFXζ-induced response or more than one human AFXζ-induced response. In another embodiment of this aspect of the present invention there are provided antagonists which are small molecules and proteins and the like which bind to human AFXζ polypeptide and regulate its biological activity. Also among preferred antagonists are molecules that bind to or interact with human AFXζ so as to inhibit an effect of human AFXζ or more than one effect of human AFXζ or which prevent expression of human AFXζ, for example, anti-sense polynucleotides which regulate transcription of the human AFXζ gene. In accordance with this aspect of the invention there are provided assays for detecting agonists and antagonists to human AFXζ which regulate human AFXζ expression and/or activity.

It is another object of the invention to provide a process for producing the aforementioned polypeptides, polypeptide fragments, variants and derivatives, fragments of the variants and derivatives, and analogs of the foregoing. In a preferred embodiment of this aspect of the invention there are provided methods for producing the aforementioned human AFXζ polypeptides comprising culturing host cells having expressibly incorporated therein a vector containing an exogenously-derived human AFXζ-encoding polynucleotide under conditions for expression of human AFXζ polypeptides in the host and then recovering the expressed polypeptide.

In accordance with another object the invention there are provided products, compositions, processes and methods that utilize the aforementioned polypeptides and polynucleotides for research, biological, clinical and therapeutic purposes, inter alia.

In certain additional preferred embodiments of this aspect of the invention there are provided antibodies against human AFXζ polypeptides and methods for their production. In certain particularly preferred embodiments in this regard, the antibodies are highly selective for human AFXζ polypeptides or portions of human AFXζ polypeptides.

In still another embodiment of the present invention there are provided compositions and methods for treating conditions mediated by human AFXζ comprising administering agonists or antagonists to human AFXζ in pharmaceutically acceptable amounts to treat a patient in need thereof.

Other objects, features, advantages and aspects of the present invention will become apparent to those of skill from the following description. It should be understood, however, that the following description and the specific examples, while indicating preferred embodiments of the invention, are given by way of illustration only. Various changes and modifications within the spirit and scope of the disclosed invention will become readily apparent to those skilled in the art from reading the following description and from reading the other parts of the present disclosure.

### BRIEF DESCRIPTION OF THE DRAWINGS

The following drawings depict certain embodiments of the invention. They are illustrative only and do not limit the invention otherwise disclosed herein.

FIG. 1. A novel isoform of human AFX. A. Relative locations of AFX primer pairs used in this study. Length of the line is not proportional to the DNA size. B. cDNA sequence 1 is taken from GenBank Accession X93996. The assumed sequence for the initiation codon is in bold. An A nucleotide in sequence I (italicized and underlined) was found absent in sequence 2, which was generated in this study and referred as AFXα. An upstream ATG (bold and italicized) was assigned as the starting codon to maintain the open reading frame. Only the differing amino acids in the corresponding protein sequences are shown. C. Sketches of AFXα and AFXζ proteins. The relevant amino acid numbers are labeled for each protein. The forkhead DNA binding domain (dotted region) is located at amino acids 97-184 of AFXα. Amino acids 58-112 of AFXα are not present in the novel isoform AFXζ. The predicted forkhead domain is shortened in AFXζ and is located at amino acids 58-129. D. Tissue distribution of AFXα and AFXζ transcripts. First-strand human cDNA samples were amplified using primers 5-3 and 3-3. PCR products were separated on 2% agarose gel and detected by Vistra green staining. The common upper band (424 bp) represents AFXα. The tissue specific 259 bp band represents the novel isoform AFXζ. Lane 1:100 bp DNA marker; lanes 2-17: heart, brain, placenta, lung, liver, skeletal muscle, kidney, pancreas, spleen, thymus, prostate, testis, ovary, small intestine, colon, leukocyte; lane 18: negative control without cDNA template.

FIG. 2. DNA binding of AFXα and AFXζ proteins. Sequences of IRS elements from the promoters of PEPCK, IGFBP-1 and G6Pase are shown in A. The numbers are relative to the start of transcription. PEPCK-a, PEPCK-b and IGFBP-1a, IGFBP-1b, are dissected sequences from PEPCK and IGFBP-1 fragments respectively. The DNA binding abilities of His-tagged AFXζ bacterial fusion protein (B) and of in vitro translated AFXζ (C) and AFXα (D) were analyzed in the electrophoretic mobility shift assay (EMSA). IRS elements from the promoters of PEPCK (lanes 2), IGFBP-1 (lanes 5) and G6Pase (lanes 8) were end-labeled and incubated with the test protein. Corresponding unlabeled IRS fragment was added as the competitor to show the specificity of each binding (lanes 3, 6, and 9). The shifted bands are indicated by arrows. Free probes are indicated by arrowheads. Lanes 1, 4 and 7 contain only probe.

FIG. 3. Transcriptional activity of AFXζ. HepG2 cells were transiently cotransfected with individual SEAP reporter construct and pcDNA6/His-AFXα or -AFXζ. CMV-β was also cotransfected to normalize for transfection efficiency. Forty-eight hours after transfection, SEAP and β-galactosidase activities were measured. The relative activation levels were standardized by comparison to the parallel transfection without pcDNA6/His-AFXα or AFXζ. All values represent the means±S.D. of 3 independent experiments. A.

Dose response of PEPCK IRS (filled circles) or PEPCK promoter (open circles) reporters by increasing amounts of AFXζ expression construct. B. Activation of various IRS-containing reporters by 0.2 μg cotransfected AFXα (open bars) or AFXζ (filled bars) expression construct.

FIG. 4. Effect of insulin on the transcriptional activity of AFXζ. HepG2 cells were transiently transfected with pcDNA6/His-AFXζ and PEPCK p-SEAP (A) or PEPCK IRS-SEAP (B). Twenty-four hours after transfection, cells were treated with insulin at the indicated concentration in fresh media and incubated for another 15-18 hours. SEAP and β-galactosidase activities were measured. The relative activation levels were standardized by comparison to the reporter activity from transfected cells without insulin treatment (A) or untreated cells transfected with the reporter alone (B). All values represent the means±S.D. of 3 independent experiments.

FIG. 5. Effect of bpV (peroxovanadate) and PKB on the transcriptional activity of AFXζ. A. HepG2 cells were transiently transfected with PEPCK p-SEAP and pcDNA6/His-AFXζ. Twenty-four hours after transfection, cells were treated with bpV at the indicated concentration in fresh media and were incubated for another 15-18 hours. The relative activation level was standardized by comparison to the reporter activity from transfection without bpV treatment. B. HepG2 cells were transiently cotransfected with the reporter constructs and pcDNA6/His-AFXζ with (filled bars) or without (open bars) the plasmid encoding myr-Akt1, which constitutively expresses active PKB. Forty-eight hours after transfection, SEAP and β-galactosidase activities were measured. The relative activation levels were standardized comparing with the activity from corresponding transfection with reporter alone. All values represent the means±S.D. of 3 independent experiments.

FIG. 6. Effect of AICAR on the transcriptional activity of AFXζ. A. HepG2 cells were transiently transfected with AFXζ and PEPCK p-SEAP (A) or PEPCK IRS-SEAP (B). Twenty-four hours after transfection, cells were treated with AICAR at indicated concentration in fresh media and incubated for another 15-18 hours. SEAP and β-galactosidase activities were measured. The relative activation levels were standardized by comparison to the activity from cells without AICAR treatment (A) or untreated cells transfected with the reporter alone (B). All values represent the means±S.D. of 3 independent experiments.

### DEFINITIONS

The following illustrative explanations are provided to facilitate understanding of certain terms used frequently herein, particularly in the examples. The explanations are provided as a convenience and are not limitative of the invention.

“Agonist” as the term is used herein, refers to any substance which augments AFXζ action by, for example, binding to the AFXζ gene product directly or by effecting enzymes and other molecules which regulate AFXζ action.

“Antagonist” as the term is used herein, refers to any substance which slows, reverses or inhibits AFXζ action.

“Identity” as the term is used herein, refers to a polynucleotide or polypeptide sequence which comprises a percentage of the same bases as a reference polynucleotide (SEQ ID NO:1) or polypeptide (SEQ ID NO:2). For example, a polynucleotide or polypeptide which is at least 90% identical to a reference polynucleotide or polypeptide, has polynucleotide bases or amino acid residues which are identical in 90% of the bases or residues which make up the reference polynucleotide or polypeptide and may have different bases or residues in 10% of the bases or residues which comprise that polynucleotide or polypeptide sequence. Exemplary algorithms for determining “identity” are the BLAST program (Basic Local Alignment Search Tool at the National Center for Biological Information (Altschul S. F. et al., J. Mol. Biol. (1990) 215(3):403410) and FAST program. “Identity” may be determined by procedures which are well-known in the art.

The term “gene” means the segment of DNA involved in producing a polypeptide chain; it includes regions preceding and following the coding region (leader and trailer) as well as intervening sequences (introns) between individual coding segments (exons).

A DNA “coding sequence of” or a “nucleotide sequence encoding” a particular protein, is a DNA sequence which is transcribed and translated into a protein when placed under the control of appropriate regulatory sequences. A “promotor sequence” is a DNA regulatory region capable of binding RNA polymerase in a cell and initiating transcription of a downstream (3′ direction) coding sequence.

The term “isolated” means that the material is removed from its original environment (e.g., the natural environment if it is naturally occurring). For example, a naturally-occurring polynucleotide or polypeptide present in a living animal is not isolated, but the same polynucleotide or polypeptide, separated from some or all of the coexisting materials in the natural system, is isolated. Such polynucleotides could be part of a vector and/or such polynucleotides or polypeptides could be part of a composition, and still be isolated in that such vector or composition is not part of its natural environment.

“Oligonucleotide(s)” refers to relatively short polynucleotides. Often the term refers to single-stranded deoxyribonucleotides, but it can refer as well to single-or double-stranded ribonucleotides, RNA:DNA hybrids and double-stranded DNAs, among others.

“Polynucleotide(s)” generally refers to any polyribonucleotide or polydeoxribonucleotide, which may be unmodified RNA or DNA or modified RNA or DNA. Thus, for instance, polynucleotides as used herein refers to, among others, single-and double-stranded DNA, DNA that is a mixture of single-and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotide as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide.

As used herein, the term polynucleotide includes DNAs or RNAs as described above that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein.

It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including simple and complex cells, inter alia.

“Polypeptides”, as used herein, includes all polypeptides as described below. The basic structure of polypeptides is well known and has been described in innumerable textbooks and other publications in the art. In this context, the term is used herein to refer to any peptide or protein comprising two or more amino acids joined to each other in a linear chain by peptide bonds. As used herein, the term refers to both short chains, which also commonly are referred to in the art as peptides, oligopeptides and oligomers, for example, and to longer chains, which generally are referred to in the art as proteins, of which there are many types.

It will be appreciated that polypeptides often contain amino acids other than the 20 amino acids commonly referred to as the 20 naturally occurring amino acids, and that many amino acids, including the terminal amino acids, may be modified in a given polypeptide, either by natural processes, such as processing and other post-translational modifications, but also by chemical modification techniques which are well known to the art. Even the common modifications that occur naturally in polypeptides are too numerous to list exhaustively here, but they are well described in basic texts and in more detailed monographs, as well as in a voluminous research literature, and they are well known to those of skill in the art. Among the known modifications which may be present in polypeptides of the present are, to name an illustrative few, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

Such modifications are well known to those of skill and have been described in great detail in the scientific literature. Several particularly common modifications, glycosylation, lipid attachment, sulfation, gamma-carboxylation of glutamic acid residues, hydroxylation and ADP-ribosylation, for instance, are described in most basic texts, such as, for instance PROTEINS - STRUCTURE AND MOLECULAR PROPERTIES, 2nd Ed., T. E. Creighton, W. H. Freeman and Company, New York (1993). The term “polypeptide” is used interchangeably herein with the terms “polypeptides” and “protein(s)”.

“Variant(s)” of polynucleotides or polypeptides, as the term is used herein, are polynucleotides or polypeptides that differ from a reference polynucleotide or polypeptide, respectively. Variants in this sense are described below and elsewhere in the present disclosure in greater detail.

(1) A polynucleotide that differs in nucleotide sequence from another, reference polynucleotide. Generally, differences are limited so that the nucleotide sequences of the reference and the variant are closely similar overall and, in many regions, identical.

As noted below, changes in the nucleotide sequence of the variant may be silent. That is, they may not alter the amino acids encoded by the polynucleotide. Where alterations are limited to silent changes of this type a variant will encode a polypeptide with the same amino acid sequence as the reference. Also as noted below, changes in the nucleotide sequence of the variant may alter the amino acid sequence of a polypeptide encoded by the reference polynucleotide. Such nucleotide changes may result in amino acid substitutions, additions, deletions, fusions and truncations in the polypeptide encoded by the reference sequence, as discussed below.

(2) A polypeptide that differs in amino acid sequence from another, reference polypeptide. Generally, differences are limited so that the sequences of the reference and the variant are closely similar overall and, in many region, identical.

A variant and reference polypeptide may differ in amino acid sequence by one or more substitutions, additions, deletions, fusions and truncations, which may be present in any combination.

### DESCRIPTION

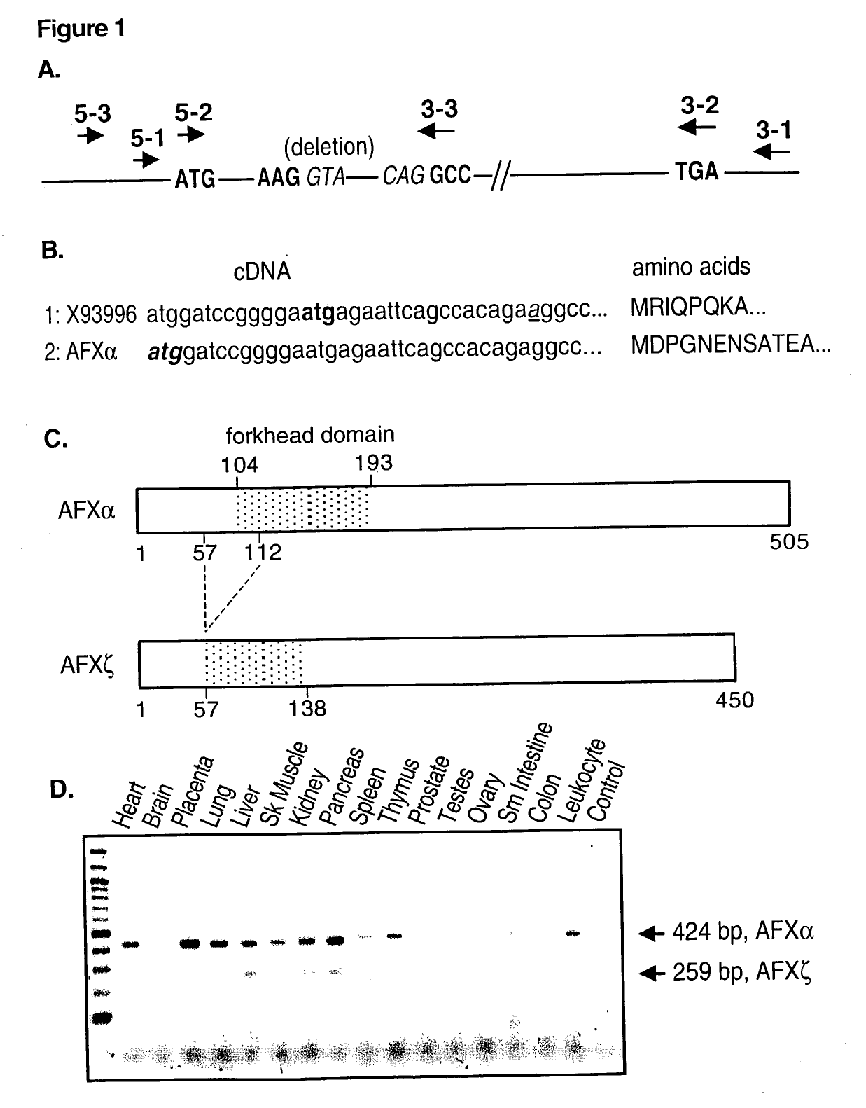

In one aspect the present invention provides a novel splice variant of the AFX gene which is designated herein as AFXζ. The AFXα protein has 505 amino acids and shares 87.7% identity with the mouse AFX protein (GenBank accession NP-061259). AFXζ protein has 450 amino acids (SEQ ID NO:2) and is encoded by an AFXζ polynucleotide as set forth in SEQ ID NO:1. AFXζ was cloned from the human liver and heart libraries. AFXζ has a detetion of 55 amino acids as compared to the AFXα protein, including the first 16 amino acids of the forkhead domain of the AFXα protein (FIG. 1C). The three characterized PKB phosphorylation sites in AFXα (26) are conserved in AFXζ as Thr-32, Ser-142, and Ser-208.

The sequence of the predicted AFXζ helix 1 mirrors the alignment of hydrophobic (Leu-54) and anionic (Glu-53 and Asp-60) amino acids that were shown to participate in the protein core and the protein-solvent interface, respectively (38). Thus, AFXζ conserves key structural aspects of helix 1.

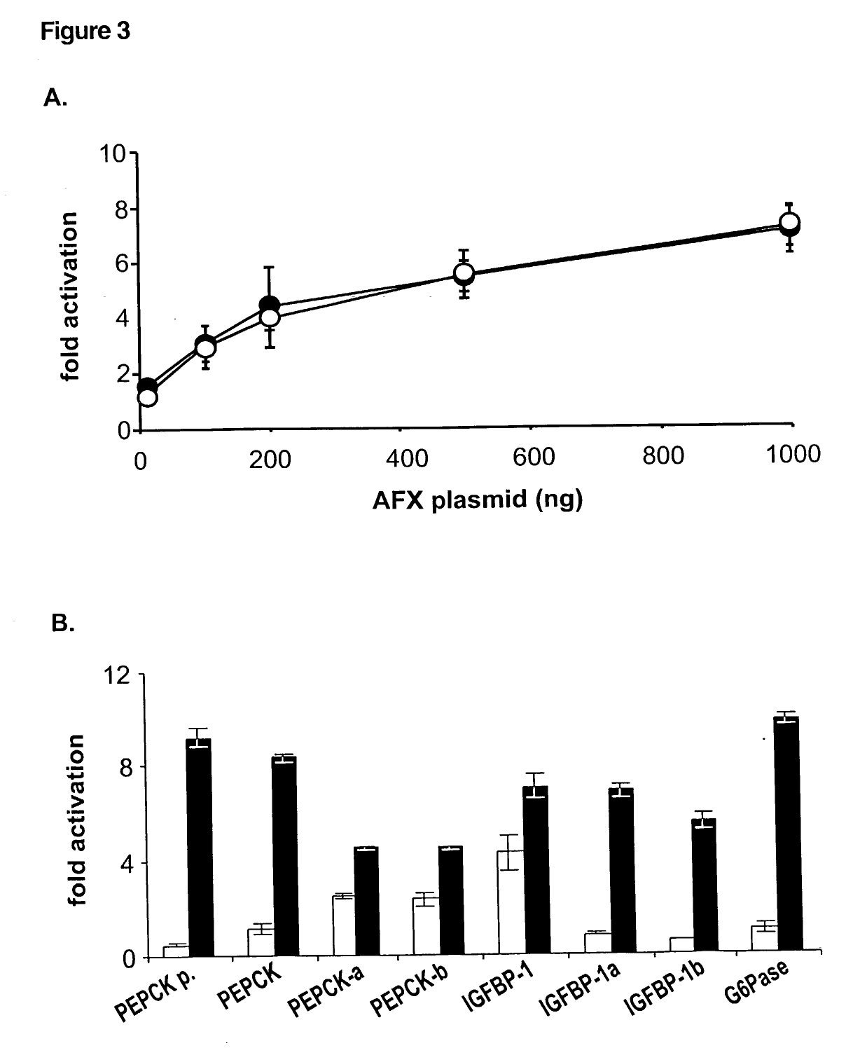

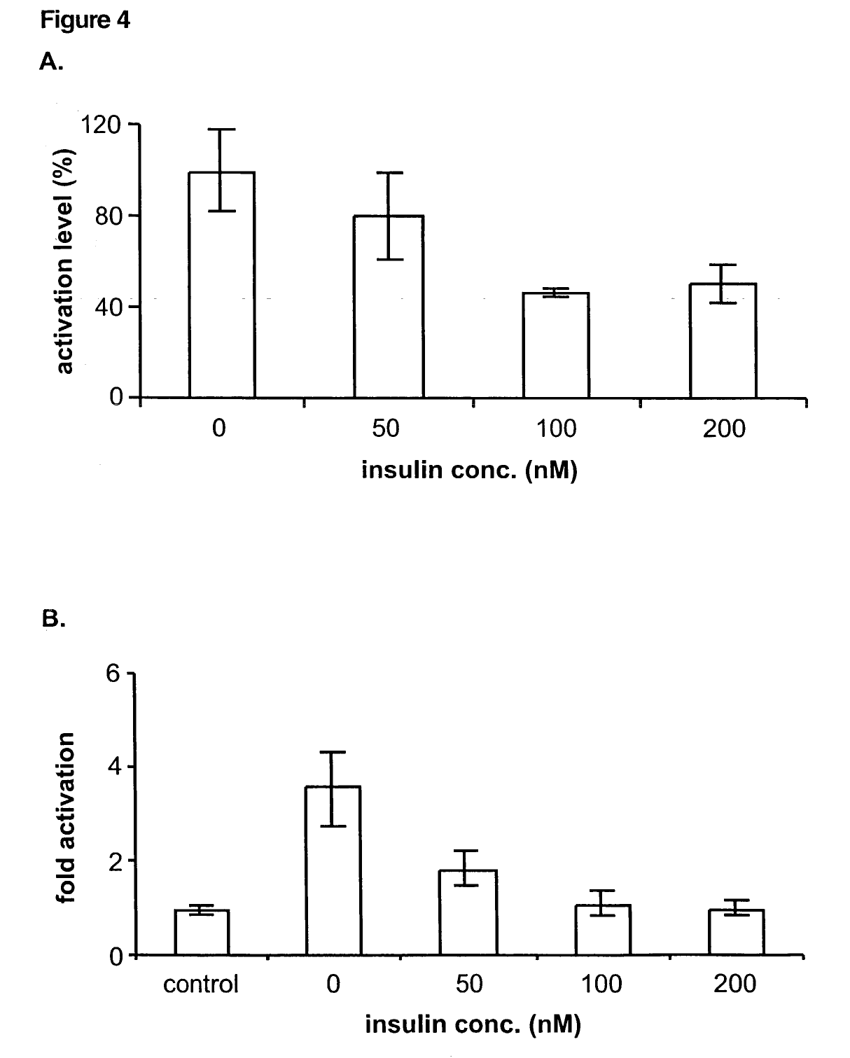

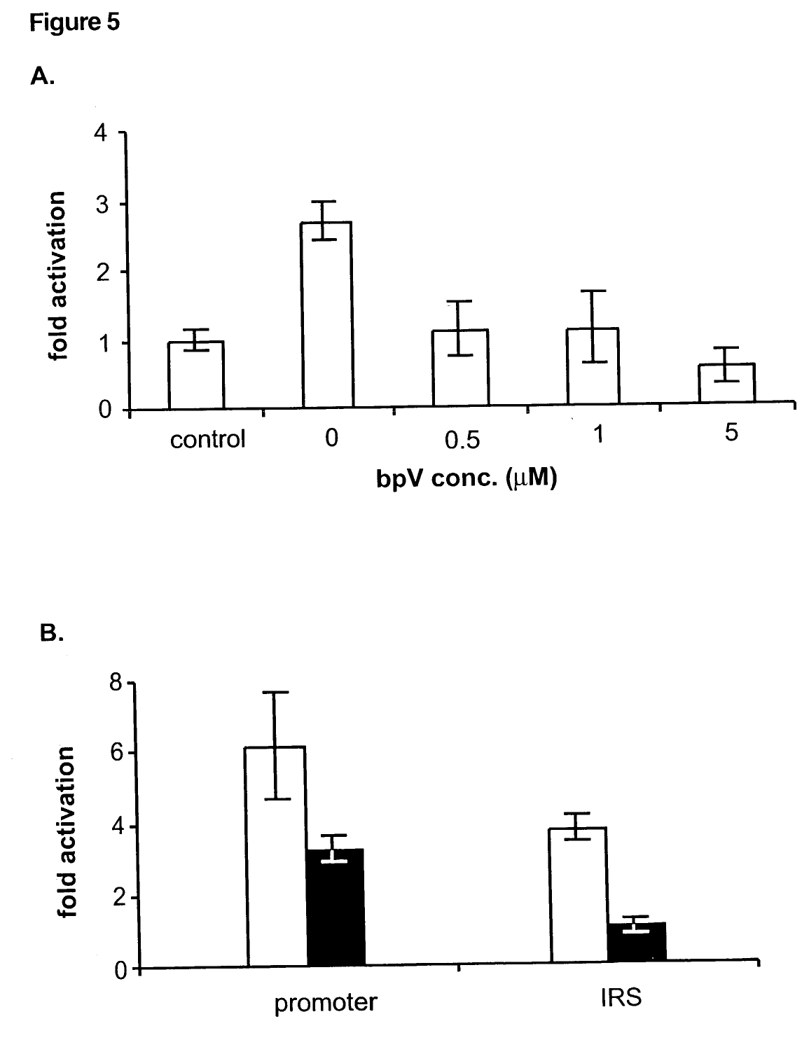

An unexpected phenomenon observed in this study is that AFXζ appears to activate the reporter transcription to higher levels than AFXα does (FIG. 3). As others have noted for similar forkhead factors, insulin repressed the transcriptional activity of AFXζ (FIG. 4). This inhibition appears to be mediated through the PI3K-PKB pathway, as treatment with peroxovanadate (bpV), an inhibitor of protein phosphatases, or overexpression of constitutively active PKB, both suppress the AFXζ-stimulated reporter activity (FIG. 5).

PCR analysis shows that AFXα is ubiquitously expressed and that AFXζ is more restricted in its expression, but that the two isoforms are coexpressed in multiple tissues (FIG. 1D). In HepG2 cells, while AFXζ activated IRSs in the IGFBP-1, PEPCK andG6Pase promoters, AFXα did not activate elements within the latter two promoters (FIG. 3). Because AFXα and the more potent AFXζ bind to similar set of DNA ligands, AFXα and AFXζ may have antagonistic transcriptional regulatory functions.

The tissue specific expression pattern of AFX isoforms reveal that AFXζ mRNA has the strongest expression in the liver, kidney and pancreas (FIG. 1D). The pancreas and the liver are crucial to glucose metabolism, and both are severely affected during the progression of diabetes. In comparison, AFXα is expressed at high levels in placenta and skeletal muscle (19). It is the inventors belief that AFXζ is a distal effector of the insulin signaling pathway.

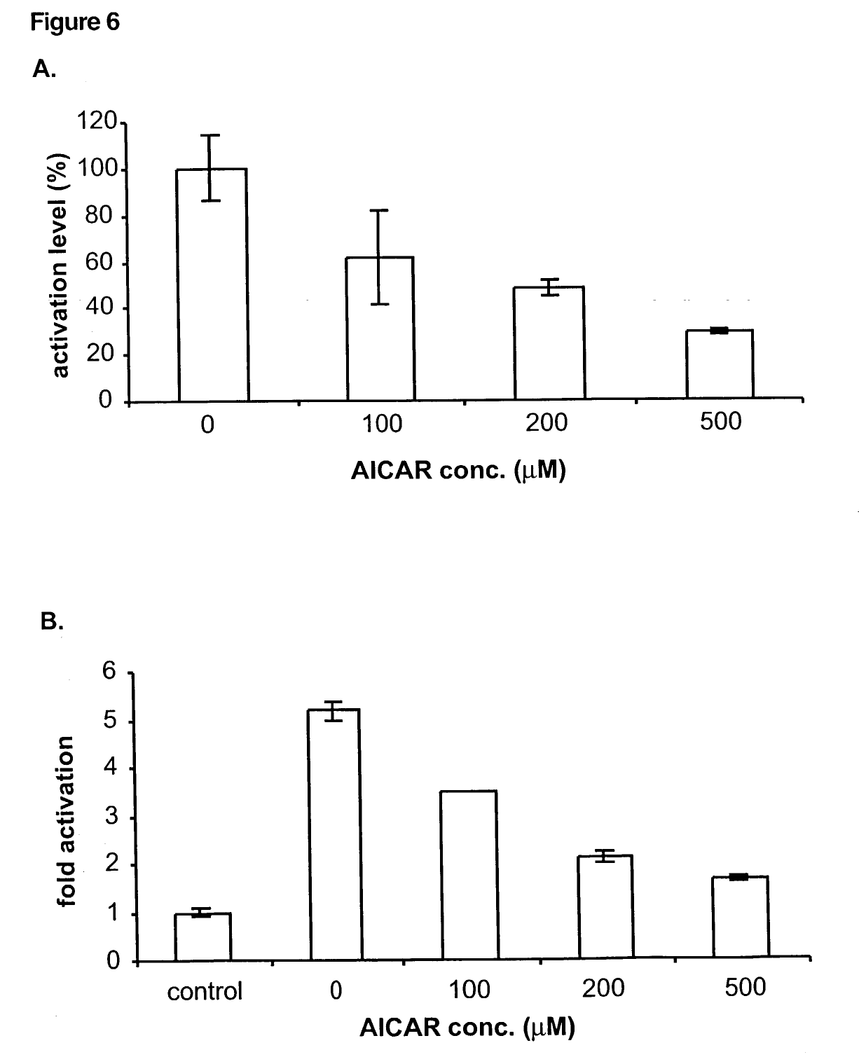

AMPK is a component of a highly conserved protein kinase cascade (39), and it influences many metabolic processes that become altered in the diabetic state (reviewed in (30)). Application of AICAR is one of the most specific methods for activating AMPK in intact cells (40). Recently, Lochhead et al. (37) showed that treatment of hepatoma cells with AICAR mimics the effect of insulin to repress the expression of the two key gluconeogenic genes, PEPCK and G6Pase. They hypothesized that AMPK and insulin may lie on distinct pathways that join at a point upstream of the two promoters (37). Treating HepG2 cells with AICAR caused a dose-dependent inhibition of AFXζ-mediated PEPCK IRS and promoter activity (FIG. 6). Therefore, AMPK appears to be involved in the regulation of the transcriptional activity of AFXζ. Winder et aL hypothesized that activation of AMPK may partially correct the metabolic perturbations in type 2 diabetes resulting from defects in the insulin signaling cascade (30). The data disclosed herein suggests that AFXζ has a profound impact in the treatment of type 2 diabetes.

AFX cDNAs were amplified by PCR from a human liver cDNA library using primer set 5-1 and 3-1 based on GenBank sequence X93996 (25) (FIG. 1A). A single A nucleotide was found to be absent 20 nucleotides downstream of the previoiusly designated initiation codon (FIG. 1 B). Correction of the sequences leads to slight changes of the predicted amino acid sequence of the protein N-terminus, as the initiation codon is shifted upstream (FIG. 1B, sequence 2). The coding region of human AFX is thus 1518 nucleotides in size and encodes 505 amino acids (FIG. 1C). The predicted forkhead domain is located at amino acids 97-184 of the sequence.

Further analyses of the cloned AFX cDNAs revealed the presence of a shorter splice variant that encodes a shorter protein lacking amino acids 58 - 112 (FIG. 1 C). This shorter sequence is AFXζ and the longer form is AFXα. The 165 bp cDNA region that is not present in AFXζ starts with a GT dinucleotide and ends with an AG. The amino acids previously identified as PKB phosphorylation sites are retained in AFXζ as Thr-32, Ser-142, and Ser-208, but the myb DNA binding homology domain of AFXα is absent in AFXζ.

PCR reactions were performed on first-strand cDNAs from different tissues using primer set 5-3 and 3-3 (FIG. 1 D) that generates a 424 bp product for AFXα and a 259 bp product for AFXζ. Among PCR amplifications from 16 different tissues, the 424 bp fragment representing AFXα was ubiquitous while the 259 bp fragment from AFXζ was more tissue-specific. The greatest expression of AFXζ was observed in the liver, kidney and pancreas (lanes 6, 8 and 9). It was also readily detectable in the heart and placenta (lanes 2 and 4). The lung, skeletal muscle, spleen, thymus and small intestine expressed AFXζ at lower levels (lanes 5, 7, 10, 11 and 15). AFXζ transcripts were not detected in samples from the brain, prostate, testis, ovary, colon and leukocyte (lanes 3, 12, 13, 14, 16 and 17).

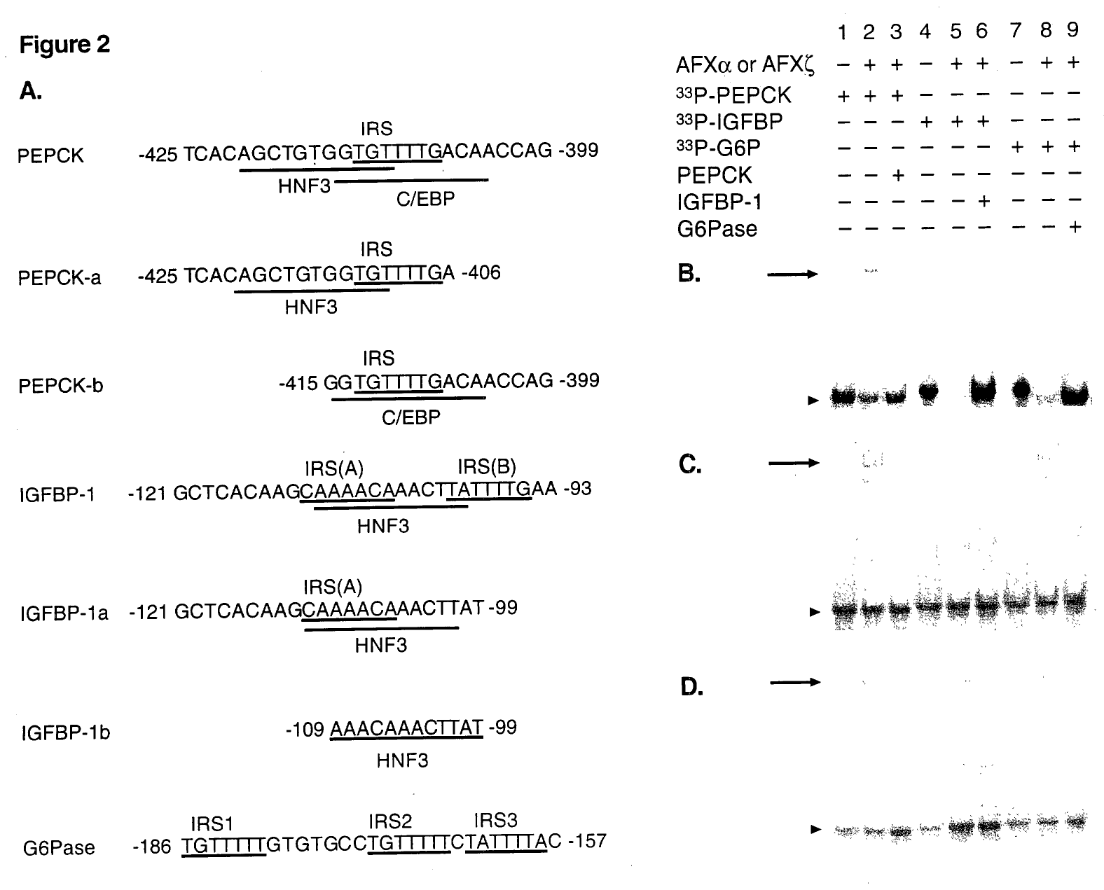

In electro-mobility shift assays, His-tagged AFXζ bacterial fusion protein bound the IRS elements from the PEPCK (FIG. 2B, lane 2), IGFBP1 (lane 5) and G6Pase (lane 8) promoters. A single DNA-protein complex was detected in each gel shift experiment. The binding was specific as it can be competed away by the addition of the corresponding unlabeled DNA fragments (lanes 3, 6 and 9). In vitro translated proteins for AFXζ(FIG. 2C)and AFXα (FIG. 2D) were also used as the protein sources for gel shift assays, and a variety of shifted bands were observed. Multiple DNA-protein complexes have been shown previously in the binding of GST-AFXα and Trx-AFXα to IRS within IGFBP-1 promoter (26,31). Therefore, AFXζ protein binds IRS fragments even though it lacks the first 16 amino acids of the forkhead domain as identified in AFXα.

Reporter constructs were generated by inserting the fragments spanning the IRS elements of the PEPCK, IGFBP-1 and G6Pase promoters (FIG. 2A) or an expanded DNA fragment of the PEPCK promoter (−458 to +43) regulatory sequence (abbreviated as PEPCK p) into the pSEAP2-promoter vector. Experiments were conducted to determine the transcriptional role of AFXζ on the expression of the reporter driven by the PEPCK IRS (-425 to -399) and the full-length promoter (−458 to +43) of PEPCK (PEPCK p). FIG. 3A shows that in HepG2 cells, AFXζ activated both PEPCK IRS and PEPCK p-enhanced reporter transcription in a dose-dependent manner and to a similar extent, suggesting that transactivation at this single IRS accounts for the entire stimulation.

The human hepatoma cell line HepG2 was cotransfected with individual reporter constructs and an expression construct for AFXα or AFXζ. AFXα expression activated the IGFBP-1 reporter approximately 4-fold (FIG. 3B). This result is consistent with previous observations that AFXα induces a 6-fold increase of reporter CAT activity when under the control of the IGFBP-1 promoter (26). AFXα failed to activate the reporters containing the IRS elements from PEPCK and G6Pase promoters or the reporter containing the extended PEPCK promoter region (−458 to +43). On the other hand, AFXζ not only stimulated IGFBP-1 reporter transcription to a high level (˜7-fold, FIG. 3B), it also increased transcription of the IRS containing PEPCK, G6Pase and extended PEPCK promoter constructs to 7- to 9-fold. Therefore, AFXζ has a broader and more potent ability to activate transcription when compared to AFXα.

The PEPCK IRS overlaps with the-consensus binding sequences for HNF3 and C/EBP; the IGFBP-1 IRS also overlaps with an HNF3 site (32-35). A number of reporter constructs were generated by inserting dissected PEPCK and IGFBP-1 IRS fragments (FIG. 2A) into the pSEAP2-promoter vector. These reporter constructs were cotransfected into HepG2 cells with either the AFXα or the AFXζ expression plasmids. Again, AFXζ activated all reporter constructs tested to a degree greater than that achieved with AFXa (FIG. 3B). It is noteworthy that AFXζ activated both PEPCK-a (IRS and HNF-3 sites) and PEPCK-b (IRS and C/EBP sites) approximately 4-fold, while the effect on PEPCK (all three sites) appeared to be higher (˜8-fold). This suggested that AFXζ, unlike AFXα, can act cooperatively with factors binding at both the HNF3 and C/EBP sites. In the case of the IGFBP-1 promoter, AFXα appeared to require both IRS sites in order to mediate transcriptional activation. Surprisingly, AFXζ could activate transcription through the HNF3 binding site alone and independently of the canonical IRSs.

In HepG2 cells, AFXζ activated the reporter transcription under the control of PEPCK promoter (FIG. 3). When the cells were treated with increasing concentrations of insulin, the induced PEPCK p-SEAP activity decreased to 50% compared with the untreated cells (FIG. 4A). Insulin also inhibited the AFXζ-induced PEPCK IRS-SEAP activity in a dose-dependent manner (FIG. 4B), decreasing reporter activity to the basal level at 200 nM insulin. Additionally, insulin repressed PEPCK IRS-SEAP in cells in which exogenous AFXζ had not been introduced.

Similar to the effect seen with insulin, peroxovanadate (bpV) reduced the AFXζ-induced PEPCK p-SEAP activity in a dose-dependent manner. The addition of bpV repressed reporter gene activity at concentrations as low as 0.5 μM (FIG. 5A). More significantly, the reporter activity was diminished by 5 μM bpV to below the basal level. There is toxicity of bpV in HepG2 cells above 5 μM as evidenced by the reduction of the control α-galactosidase activity.

A plasmid encoding constitutively active PKB was cotransfected with an AFXζ expression construct and the PEPCK p-SEAP reporter construct to assess the role of the PI3K/PKB insuling signaling pathway in the suppresion of AFXζ transactivation. Expression of constitutively active PKB mimicked the effect of insulin by inhibiting the transcription of IGFBP-1 (26,29,36). FIG. 5B shows that PKB expression repressed transcription from the PEPCK promoter construct and suppressed the transcriptional activity of AFXζ on PEPCK-IRS to the basal level (FIG. 5B).

As shown above, the treatment of HepG2 cells with peroxovanadate (FIG. 5A) inhibited the reporter activity of PEPCK IRS to a greater extent than with maximally effective concentrations of insulin (FIG. 4) or with the overexpression of constitutively active PKB (FIG. 5B). AICAR is used to treat HepG2 cells that had been cotransfected with AFXζ expression construct and PEPCK IRS- or PEPCK p-SEAP reporter constructs. As shown in FIG. 6, increasing concentrations of AICAR progressively inhibited AFXζ-induced SEAP activity. At 200 μM, AICAR reduced the transcriptional activity of AFXζ on the PEPCK promoter to 48% of untreated cells (FIG. 6A). A similar reduction by AICAR treatment is also observed when PEPCK IRS-SEAP is used as the reporter. At 200 μM AICAR, the induced SEAP activity was decreased to 20% of the stimulation ascribed to transfected AFXζ(FIG. 6B). The data show that AICAR mimics the insulin effect in repressing AFXζ-induced gene expression and that this effect can be mediated through IRS.

In accordance with the above there are provided isolated polynucleotides that encode the AFXζ polypeptide having the deduced amino acid sequence of SEQ ID NO:2. Also among preferred embodiments of this aspect of the present invention are polynucleotides comprising fragments of the polynucleotide sequence set out in SEQ ID NO:1, and fragments of variants and derivatives of the polynucleotide sequence of SEQ ID NO:1.

In this regard a fragment is a polynucleotide having a nucleotide sequence that entirely is the same as part but not all of the nucleotide sequence of the aforementioned AFXζ polynucleotides and variants or derivatives thereof.

Using the information provided herein, such as the polynucleotide sequence set out in SEQ ID NO:1, a polynucleotide of the present invention encoding a human AFXζ polypeptide may be obtained using standard cloning and screening procedures, such as those for cloning cDNAs using mRNA from cells of human tissue as starting material. The cDNA sequence contains an open reading frame encoding a protein of about 450 amino acid residues.

Polynucleotides of the present invention may be in the form of RNA, such as mRNA, or in the form of DNA, including, for instance, cDNA obtained by cloning or produced by chemical synthetic techniques or by a combination thereof. The DNA may be double-stranded or single-stranded. Single-stranded DNA may be the coding strand, also known as the sense strand, or it may be the non-coding strand, also referred to as the anti-sense strand.

The coding sequence which encodes the polypeptide may be identical to the coding sequence of the polynucleotide shown in SEQ ID NO:1. It also may be a polynucleotide with a different sequence, which, as a result of the redundancy (degeneracy) of the genetic code, encodes the polypeptide of SEQ ID NO:2.

Polynucleotides of the present invention which encode the polypeptide of SEQ ID NO:2 may include, but are not limited to the coding sequence for the mature splice variant form of the polypeptide, by itself; the coding sequence of the mature polypeptide, together with additional, non-coding sequences, including for example, but not limited to introns and non-coding 5′ and 3′ sequences, such as the transcribed, non-translated sequences that play a role in transcription, mRNA processing, including splicing and polyadenylation signals, for example, ribosome binding and stability of mRNA; additional coding sequence which codes for additional amino acids, such as those which provide additional functionalities. Thus, for instance, the polypeptide may be fused to a marker sequence, such as a peptide, which facilitates purification of the fused polypeptide.

The present invention further relates to variants of the hereinabove described polynucleotides which encode for fragments, analogs and derivatives of the polypeptide having the deduced amino acid sequence of SEQ ID NO:2. A variant of the polynucleotide may be a naturally occurring variant such as a naturally occurring allelic variant, or it may be a variant that is not known to occur naturally. Such non-naturally occurring variants of the polynucleotide may be made by mutagenesis techniques, including those applied to polynucleotides, cells or organisms.

Among variants in this regard are variants that differ from the aforementioned polynucleotides by nucleotide substitutions, deletions or additions. The substitutions, deletions or additions may involve one or more nucleotides. The variants may be altered in coding or non-coding regions or both. Alterations in the coding regions may produce conservative or non-conservative amino acid substitutions, deletions or additions.

Further particularly preferred in this regard are polynucleotides encoding AFXζ variants, analogs, derivatives and fragments, and variants, analogs and derivatives of the fragments, which have the amino acid sequence of the AFXζ polypeptide of SEQ ID NO:2 in which several, a few, 5 to 10, 1 to 5, 1 to 3, 2, 1 or no amino acid residues are substituted, deleted or added, in any combination. Especially preferred among these are silent substitutions, additions and deletions, which do not alter the properties and activities of AFXζ. Also especially preferred in this regard are conservative substitutions. Highly preferred are polynucleotides encoding polypeptides having the amino acid sequence of SEQ ID NO:2 without substitutions.

Further preferred embodiments of the invention are polynucleotides that are at least 90% identical to a polynucleotide encoding the AFXζ polypeptide having the amino acid sequence set out in SEQ ID NO:2, and polynucleotides which are complementary to such polynucleotides. Alternatively, most highly preferred are polynucleotides that comprise a region that is at least 95% identical to a polynucleotide encoding the AFXζ polypeptide and polynucleotides complementary thereto. Furthermore, those with at least 97% are highly preferred among those with at least 95%, and among these those with at least 98% and at least 99% are particularly highly preferred, with at least 99% being the more preferred.

Particularly preferred embodiments in this respect, moreover, are polynucleotides which encode polypeptides which retain substantially the same biological function or activity as the polypeptide encoded by the cDNA of SEQ ID NO:1.

The present invention further relates to polynucleotides that hybridize to the herein above-described sequences under stringent conditions. As herein used, the term “stringent conditions” includes moderate or severe stringency. Conditions of moderate stringency, as defined by Sambrook et al. Molecular Cloning: A Laboratory manual, 2 ed. Vol.1, pp.101-104, Cold Spring Harbor Laboratory Press, (1989), include use of a prewashing solution of 5 X SSC, 0.5% SDS, 1.0 mM EDTA (pH 8.0) and hybridization condition of about 55° C., 5 X SSC, overnight. Conditions of severe stringency include higher temperatures of hybridization and washing. The skilled artisan will recognize that the temperature and wash solution salt concentration may be adjusted as necessary according to factors such as the length of the probe. In this regard, the present invention especially relates to polynucleotides which hybridize under stringent conditions to the herein above-described polynucleotides.

A direct submission of the human AFXζ cDNA and protein was made on May 22, 2001 to GenBank in Rockville, Md., U.S.A. and assigned GenBank Acession number AF384029.

The information contained in the submission will be irrevocably and without restriction or condition released to the public upon the issuance of a patent. The submission is provided merely as convenience to those of skill in the art and is not an admission that a submission is required for enablement, such as that required under 35 U.S.C. §112.

The sequence of the polynucleotides contained in the submission, as well as the amino acid sequence of the polypeptide encoded thereby, are controlling in the event of any conflict with any description of sequences herein.

In another aspect of the present invention, AFXζ polypeptides which have the deduced amino acid sequence of SEQ ID NO:2 are provided, as well as fragments, derivatives and analogs of such polypeptide. The terms “fragment,” “derivative” and “analog” when referring to the polypeptide of SEQ ID NO:2 means a polypeptide which retains essentially the same biological function or activity as such polypeptide. The polypeptide of the present invention may be a recombinant polypeptide, a natural polypeptide or a synthetic polypeptide, preferably a recombinant polypeptide.

The fragments, derivatives or analogs of the polypeptide of SEQ ID NO:2 may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the polypeptide is fused with another compound, such as a compound to increase the half-life of the polypeptide (for example, polyethylene glycol), or (iv) one in which the additional amino acids are fused to the polypeptide for purification of the polypeptide or a proprotein sequence. Such fragments, derivatives and analogs are deemed to be within the scope of those skilled in the art from the teachings herein.

The polypeptides of the present invention include the polypeptide of SEQ ID NO:2 as well as polypeptides which have at least 90% similarity (preferably at least 90% identity) to the polypeptide of SEQ ID NO:2 and more preferably at least and more preferably at least 95% similarity (still more preferably at least 95% identity) and even more preferably at least 97% similarity (still more preferably at least 97% identity) and most preferably at least 99% similarity (still more preferably at least 99% identity) to the polypeptide of SEQ ID NO:2.

Among preferred variants are those that vary from a reference by conservative amino acid substitutions. Such substitutions are those that substitute a given amino acid by another amino acid of like character. Conservative substitutions are the replacements, one for another, among the aliphatic amino acids Ala, Val, Leu and Ile; interchange of the hydroxyl residues Ser and Thr, exchange of the acidic residues Asp and Glu, substitution between the amide residues Asn and Gln, exchange of the basic residues Lys and Arg and replacements among the aromatic residues Phe, Tyr.

Also among preferred embodiments of this aspect of the present invention are polypeptides comprising fragments of AFXζ, most particularly fragments of the AFXζ having the amino acid set out in SEQ ID NO:2, and fragments of variants and derivatives of the AFXζ of SEQ ID NO:2. In this regard a fragment is a polypeptide having an amino acid sequence that entirely is the same as part but not all of the amino acid sequence of the aforementioned AFXζ polypeptides and variants or derivatives thereof.

Such fragments may be “free-standing,” i.e., not part of or fused to other amino acids or polypeptides, or they may be comprised within a larger polypeptide of which they form a part or region. When comprised within a larger polypeptide, the presently discussed fragments most preferably form a single continuous region. However, several fragments may be comprised within a single larger polypeptide. For instance, certain preferred embodiments relate to a fragment of a AFXζ polypeptide of the present comprised within a precursor polypeptide designed for expression in a host and having heterologous pre and pro-polypeptide regions fused to the amino terminus of the AFXζ fragment and an additional region fused to the carboxyl terminus of the fragment. Therefore, fragments in one aspect of the meaning intended herein, refers to the portion or portions of a fusion polypeptide or fusion protein derived from AFXζ.

In this context about includes the particularly recited range and ranges larger or smaller by several, a few, 5, 4, 3, 2 or 1 amino acids at either extreme or at both extremes. Highly preferred in this regard are the recited ranges plus or minus as many as 5 amino acids at either or at both extremes. Particularly highly preferred are the recited ranges plus or minus as many as 3 amino acids at either or at both the recited extremes. Especially particularly highly preferred are ranges plus or minus 1 amino acid at either or at both extremes or the recited ranges with no additions or deletions.

Vectors, Host Cells and Expression

The present invention also relates to vectors which include polynucleotides of the present invention, host cells which are genetically engineered with vectors of the invention and the production of polypeptides of the invention by recombinant techniques.

Host cells can be genetically engineered to incorporate polynucleotides and express polypeptides of the present invention. For instance, polynucleotides may be introduced into host cells using well known techniques of infection, transduction, transfection, transfection and transformation. The polynucleotides may be introduced alone or with other polynucleotides. Such other polynucleotides may be introduced independently, co-introduced or introduced joined to the polynucleotides of the invention.

Thus, for instance, polynucleotides may be transfected into host cells with another, separate, polynucleotide encoding a selectable marker, using standard techniques for co-transfection and selection in, for instance, mammalian cells. In this case the polynucleotides generally will be stably incorporated into the host cell genome.

Alternatively, the polynucleotides may be joined to a vector containing a selectable marker for propagation in a host. The vector construct may be introduced into host cells by the aforementioned techniques. Generally, a plasmid vector is introduced as DNA in a precipitate, such as a calcium phosphate precipitate, or in a complex with a charged lipid. Electroporation also may be used to introduce polynucleotides into a host. If the vector is a virus, it may be packaged in vitro or introduced into a packaging cell and the packaged virus may be transduced into cells. A wide variety of techniques suitable for making polynucleotides and for introducing polynucleotides into cells in accordance with this aspect of the invention are well known and routine to those of skill in the art. Such techniques are reviewed at length in Sambrook et al. cited above, which is illustrative of the many laboratory manuals that detail these techniques.

In accordance with this aspect of the invention the vector may be, for example, a plasmid vector, a single or double-stranded phage vector, a single or double-stranded RNA or DNA viral vector. Such vectors may be introduced into cells as polynucleotides, preferably DNA, by well known techniques for introducing DNA and RNA into cells. The vectors, in the case of phage and viral vectors also may be and preferably are introduced into cells as packaged or encapsidated virus by well known techniques for infection and transduction. Viral vectors may be replication competent or replication defective. In the latter case viral propagation generally will occur only in complementing host cells.

Preferred among vectors, in certain respects, are those for expression of polynucleotides and polypeptides of the present invention. Generally, such vectors comprise cis-acting control regions effective for expression in a host operatively linked to the polynucleotide to be expressed. Appropriate trans-acting factors either are supplied by the host, supplied by a complementing vector or supplied by the vector itself upon introduction into the host.

In certain preferred embodiments in this regard, the vectors provide for specific expression. Such specific expression may be inducible expression or expression only in certain types of cells or both inducible and cell-specific. Particularly preferred among inducible vectors are vectors that can be induced for expression by environmental factors that are easy to manipulate, such as temperature and nutrient additives. A variety of vectors suitable to this aspect of the invention, including constitutive and inducible expression vectors for use in prokaryotic and eukaryotic hosts, are well known and employed routinely by those of skill in the art.

The engineered host cells can be cultured in conventional nutrient media, which may be modified as appropriate for, inter alia, activating promoters, selecting transformants or amplifying genes. Culture conditions, such as temperature, pH and the like, previously used with the host cell selected for expression generally will be suitable for expression of polypeptides of the present invention as will be apparent to those of skill in the art.

A great variety of expression vectors can be used to express a polypeptide of the invention. Such vectors include chromosomal, episomal and virus-derived vectors e.g., vectors derived from bacterial plasmids, from bacteriophage, from yeast episomes, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids, all may be used for expression in accordance with this aspect of the present invention. Generally, any vector suitable to maintain, propagate or express polynucleotides to express a polypeptide in a host may be used for expression in this regard.

The appropriate DNA sequence may be inserted into the vector by any of a variety of well-known and routine techniques. In general, a DNA sequence for expression is joined to an expression vector by cleaving the DNA sequence and the expression vector with one or more restriction endonucleases and then joining the restriction fragments together using T4 DNA ligase. Procedures for restriction and ligation that can be used to this end are well known and routine to those of skill. Suitable procedures in this regard, and for constructing expression vectors using alternative techniques, which also are well known and routine to those skill, are set forth in great detail in Sambrook et al. cited elsewhere herein.

The DNA sequence in the expression vector may be operatively linked to appropriate expression control sequence(s), including, for instance, a promoter to direct mRNA transcription. Representatives Qf such promoters include the phage lambda PL promoter, the E. coli lac, trp and tac promoters, the SV40 early and late promoters and promoters of retroviral LTRs, to name just a few of the well-known promoters. It will be understood that numerous promoters not mentioned are suitable for use in this aspect of the invention are well known and readily may be employed by those of skill in the manner illustrated by the discussion and the examples herein.

In general, expression constructs will contain sites for transcription initiation and termination, and, in the transcribed region, a ribosome binding site for translation. The coding portion of the transcripts expressed by the constructs will include a translation initiating AUG at the beginning and a termination codon appropriately positioned at the end of the polypeptide to be translated.

In addition, the constructs may contain control regions that regulate as well as engender expression. Generally, in accordance with many commonly practiced procedures, such regions will operate by controlling transcription, such as repressor binding sites and enhancers, among others.

Vectors for propagation and expression generally will include selectable markers. Such markers also may be suitable for amplification or the vectors may contain additional markers for this purpose. In this regard, the expression vectors preferably contain one or more selectable marker genes to provide a phenotypic trait for selection of transformed host cells. Preferred markers include dihydrofolate reductase or neomycin resistance for eukaryotic cell culture, and tetracycline, theomycin, kanamycin or ampicillin resistance genes for culturing E. coli and other bacteria.

The vector containing the appropriate DNA sequence as described elsewhere herein, as well as an appropriate promoter, and other appropriate control sequences, may be introduced into an appropriate host using a variety of well known techniques suitable to expression therein of a desired polypeptide. Representative examples of appropriate hosts include bacterial cells, such as E. coli, Streptomyces and Salmonella typhimurium cells; fungal cells, such as yeast cells; insect cells such as Drosophila S2 and Spodoptera Sf9 cells; animal cells such as CHO, COS and Bowes melanoma cells; and plant cells. Hosts for a great variety of expression constructs are well known, and those of skill will be enabled by the present disclosure readily to select a host for expressing a polypeptides in accordance with this aspect of the present invention.

Various mammalian cell culture systems can be employed for expression, as well. Examples of mammalian expression systems include the COS-7 lines of monkey kidney fibroblast, described in Gluzman et al., Cell 23: 175 (1981). Other cell lines capable of expressing a compatible vector include for example, the C127, 3T3, CHO, HeLa, human kidney 293 and BHK cell lines.

More particularly, the present invention also includes recombinant constructs, such as expression constructs, comprising one or more of the sequences described above. The constructs comprise a vector, such as a plasmid or viral vector, into which such a sequence of the invention has been inserted. The sequence may be inserted in a forward or reverse orientation. In certain preferred embodiments in this regard, the construct further comprises regulatory sequences, including, for example, a promoter, operably linked to the sequence. Large numbers of suitable vectors and promoters are known to those of skill in the art, and there are many commercially available vectors suitable for use in the present invention.

The following vectors, which are commercially available, are provided by way of example. Among preferred eukaryotic vectors are pWLNEO, pSV2CAT, pOG44, pXT1 and pSG available from Stratagene; pcDNA3 available from Invitrogen; and pSVK3, pBPV, PMSG and pSVL available from Pharmacia. These vectors are listed solely by way of illustration of the many commercially available and well known vectors that are available to those of skill in the art for use in accordance with this aspect of the present invention. It will be appreciated that any other plasmid or vector suitable for, for example, introduction, maintenance, propagation or expression of a polynucleotide or polypeptide of the invention in a host may be used in this aspect of the invention.

Generally, recombinant expression vectors will include origins of replication, a promoter derived from a highly-expressed gene to direct transcription of a downstream structural sequence, and a selectable marker to permit isolation of vector containing cells after exposure to the vector.

The present invention also relates to host cells containing the above-described constructs discussed above. The host cell can be a higher eukaryotic cell, such as a mammalian cell, or a lower eukaryotic cell, such as a yeast cell, or the host cell can be a prokaryotic cell, such as a bacterial cell.

Constructs in host cells can be used in a conventional manner to produce the gene product encoded by the recombinant sequence. Alternatively, the polypeptides of the invention can be synthetically produced by conventional peptide synthesizers.

Proteins can be expressed in mammalian cells, yeast, bacteria, or other cells under the control of appropriate promoters. Cell-free translation systems can also be employed to produce such proteins using RNAs derived from the DNA constructs of the present invention. Appropriate cloning and expression vectors for use with prokaryotic and eukaryotic hosts are described by Sambrook et al., Molecular Cloning: A Laboratory Manual, 2nd Ed., Cold Spring Harbor Laboratory Press, Cold Spring Harbor, N.Y. (1989).

The polypeptide may be expressed in a modified form, such as a fusion protein, and may include additional heterologous functional regions. Thus, for instance, a region of additional amino acids, particularly charged amino acids, may be added to the N-terminus of the polypeptide to improve stability and persistence in the host cell, during purification or during subsequent handling and storage. Also, regions may be added to the polypeptide to facilitate purification. Such regions may be removed prior to final preparation of the polypeptide. The addition of peptide moieties to polypeptides to engender excretion, to improve stability and to facilitate purification, among others, are familiar and routine techniques in the art.

Following transformation of a suitable host strain and growth of the host strain to an appropriate cell density, where the selected promoter is inducible it is induced by appropriate means (e.g., temperature shift or exposure to chemical inducer) and cells are cultured for an additional period. Cells typically then are harvested by centrifugation, disrupted by physical or chemical means, and the resulting crude extract retained for further purification.

Microbial cells employed in expression of proteins can be disrupted by any convenient method, including freeze-thaw cycling, sonication, mechanical disruption, or use of cell lysing agents, such methods are well know to those skilled in the art.

The AFXζ polypeptide can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography (HPLC) is employed for purification. Well known techniques for refolding protein may be employed to regenerate active conformation when the polypeptide is denatured during isolation and or purification.

AFXζ polynucleotides and polypeptides may be used in accordance with the present invention for a variety of applications, particularly the AFXζ polynucleotide and polypeptide may be used as a target to identify compounds which inhibit or retard expression of AFXζ polynucleotides or biological activity of AFXζ polypeptides to treat and/or prevent arthritis, cancer, cancer metastases, and diseases caused by cellular apoptosis including but not limited to Parkinson's disease, Alzheimer's disease and Huntington's chorea. Additional applications relate to diagnosis and to treatment of disorders of cells, tissues and organisms. These aspects of the invention are illustrated further by the following discussion.

Antibodies

The polypeptides, their fragments or other derivatives, or analogs thereof, or cells expressing them can be used as an immunogen to produce antibodies thereto as. These antibodies can be, for example, polyclonal or monoclonal antibodies. The present invention also includes chimeric, single chain, and humanized antibodies, as well as Fab fragments, or the product of an Fab expression library. Various procedures known in the art may be used for the production of such antibodies and fragments.

Antibodies generated against the polypeptides corresponding to a sequence of the present invention can be obtained by direct injection of the polypeptides or a fragment thereof into an animal or by administering the polypeptides to an animal, preferably a nonhuman. The antibody so obtained will then bind the polypeptides itself. In this manner, even a sequence encoding only a fragment of the polypeptides can be used to generate antibodies binding the whole native polypeptides. Such antibodies can then be used to isolate the polypeptide from tissue expressing that polypeptide. The antibodies may also be used to bind a soluble form of the polypeptide and therefore render it ineffective to perform its intended biological function.

For preparation of monoclonal antibodies, any technique which provides antibodies produced by continuous cell line cultures can be used. Examples include the hybridoma technique (Kohler, G. and Milstein, C., Nature 256: 495497 (1975), the trioma technique, the human B-cell hybridoma technique (Kozbor et al., Immunology Today 4: 72 (1983) and the EBV-hybridoma technique to produce human monoclonal antibodies (Cole et al., pg. 77-96 in Monoclonal Antibodies and Cancer Therapy, Alan R. Liss, Inc. (1985).

Techniques described for the production of single chain antibodies (U.S. Pat. No.4,946,778) can be adapted to produce single chain antibodies to immunogenic polypeptide products of this invention. Also, transgenic mice, or other organisms such as other mammals, may be used to express humanized antibodies to immunogenic polypeptide products of this invention.

The above-described antibodies may be employed to isolate or to identify clones expressing the polypeptide or purify the polypeptide of the present invention by attachment of the antibody to a solid support for isolation and/or purification by affinity chromatography. Further, the antibodies may be employed as antagonists as described below.

Antagonists

The invention also provides a method of identifying antagonists which reduce or inhibit the action of human AFXζ protein. For example, a cellular compartment, such as a membrane or a preparation thereof, such as a membrane-preparation, may be prepared from a cell that expresses a molecule that binds human AFXζ protein, such as a molecule of a signaling or regulatory pathway modulated by human AFXζ protein. The preparation is incubated with labeled human AFXζ protein in the absence or the presence of a candidate molecule which may be a human AFXζ antagonist. The ability of the candidate molecule to bind the binding molecule is reflected in decreased binding of the labeled ligand. Molecules which bind gratuitously, i.e., without inducing the effects of human AFXζ on binding the human AFXζ binding molecule, are most likely to be good antagonists.

Human AFXζ-like effects of potential antagonists may by measured, for instance, by determining activity of a second messenger system following interaction of the candidate molecule with a cell or appropriate cell preparation, and comparing the effect with that of human AFXζ or molecules that elicit the same effects as human AFXζ. Second messenger systems that may be useful in this regard include but are not limited to AMP guanylate cyclase, ion channel or phosphoinositide hydrolysis second messenger systems.

Another example of an assay for identifying human AFXζ antagonists is a competitive assay that combines human AFXζ and a potential antagonist with an AFXζ substrate identified. Appropriate conditions for a competitive inhibition assay including optimal kinetic parameters are first determined with AFXζ and an appropriate substrate. Human AFXζ activity is determined by measuring the disappearance of the substrate using HPLC. The same assay is then performed in the presence of a potential inhibitor or antagonist and the rate of disappearance of the substrate is again measured to determine the effectiveness of the candidate antagonist.

Potential antagonists include small organic molecules, peptides, polypeptides and antibodies which can pass through the cell membrane and bind to a polypeptide of the invention and thereby inhibit or extinguish its activity. Additionial antagonists include AICAR or insulin genes. Other potential antagonists include antisense molecules for preventing expression of the AFXζ gene. Antisense technology can be used to control gene expression through antisense DNA or RNA or through triple-helix formation. Antisense techniques are discussed, for example, in - Okano, J. Neurochem. 56: 560 (1991); Oligodeoxynucleotides as Antisense Inhibitors of Gene Expression, CRC Press, Boca Raton, Fla. (1988). Triple helix formation is discussed in, for instance Lee et al., Nucleic Acids Research 6: 3073 (1979); Cooney et al., Science 241: 456 (1988); and Dervan et al., Science 251: 1360 (1991). The methods are based on binding of a polynucleotide to a complementary DNA or RNA. For example, the 5′ coding portion of a polynucleotide that encodes the mature polypeptide of the present invention may be used to design an antisense RNA oligonucleotide of from about 10 to 40 base pairs in length. A DNA oligonucleotide is designed to be complementary to a region of the gene (or promotor) involved in transcription thereby preventing transcription and the production of human AFXζ. The antisense RNA oligonucleotide hybridizes to the mRNA in vivo and blocks translation of the mRNA molecule into human AFXζ polypeptide. The oligonucleotides described above can also be delivered to cells such that the antisense RNA or DNA may be expressed in vivo to inhibit production of human AFXζ.

The antagonists may be employed in a composition with a pharmaceutically acceptable carrier, e.g., as hereinafter described. The antagonists may be employed for instance to treat and/or prevent diseases or conditions including but not limited to insulin resistance, hyperglycemia, hypoglycemia, hepatic gluconeogensis, hypercholesteremia, obesity, type 2 diabetes, impaired glucose tolerance, disorders related to Syndrome X including hypertension, obesity, insulin resistance, coronary artery disease, glomerulonephritis, glomerulosclerosis, nephrotic syndrome and hypertensive nephrosclerosis.

Agonists

In accordance with another aspect of the present invention, there are provided AFXζ agonists. Among preferred agonists are small molecules that mimic AFXζ protein, that bind to AFXζ binding molecules, and that elicit or augment AFXζ induced responses. Also among preferred agonists are molecules that interact with AFXζ or AFXζ polypeptides, or with other modulators of AFXζ activities, and thereby potentiate or augment an effect of AFXζ or more than one effect of AFXζ.

The agonists may be employed in a composition with a pharmaceutically acceptable carrier, e.g., as hereinafter described. The agonists may be employed for instance to treat and/or prevent diseases or conditions including but not limited to insulin resistance, hyperglycemia, hypoglycemia, hepatic gluconeogensis, hypercholesteremia, obesity, type 2 diabetes, impaired glucose tolerance, disorders related to Syndrome X including hypertension, obesity, insulin resistance, coronary artery disease, glomerulonephritis, glomerulosclerosis, nephrotic syndrome and hypertensive nephrosclerosis.

Methods for identifving both agonists and antagonists of AFXζ regulated genes

Expression constructs for AFXζ and IRS-containing plasmids may be used to devise various reporter gene assays to find both compounds and genes that affect AFXζ-regulated genes such as PEPCK, IGFBP-1, and G6Pase. As one example, an AFXζ expression construct and an IRS-containing reporter construct, when transfected into a eukaryotic cell line, will give a certain signal. Exposure of the cells to test compounds may reveal that certain compounds reduce AFXζ-dependent reporter gene activation in a manner similar to that seen with AICAR. Such compounds may be employed to repress hepatic gluconeogenesis in a diabetic individual. Alternatively, compounds that augment AFXζ function may be employed to increase the activity of other potential target genes such as ABCA1 in dyslipidemic patients and in people with atherosclerosis.

In another embodiment of this aspect of the present invention, a similar pairing of an AFXζ expression construct and an IRS-containing reporter gene construct may be used to find genes that modulate, either positively or negatively, the reporter. Such genes are likely to be participants in insulin signal transduction pathways or AMPK-dependent signaling pathways, and as such, may be valuable drug targets. In one embodiment, standard expression cloning methodologies could be used to introduce expression constructs for many anonymous genes individually into cells already containing AFXζ and an IRS-containing reporter. Such reporter genes may include luciferase, β-galactosidase, green fluorescent protein, and others. Some genes will increase reporter gene activity, and others may decrease it, and these changes may be detected in enzymatic or fluorescent assays, including fluorescent activated cell sorting.

Compositions

The invention also relates to compositions comprising the antagonists and agonists. Thus, the antagonists of the present invention may be employed in combination with a non-sterile or sterile carrier or carriers for use with cells, tissues or organisms, such as a pharmaceutical carrier suitable for administration to a subject. Such compositions comprise, for instance, a media additive or a therapeutically effective amount of an agonist or antagonist of the invention and a pharmaceutically acceptable carrier or excipient. Such carriers may include, but are not limited to, saline, buffered saline, dextrose, water, glycerol, ethanol and combinations thereof. The formulation should suit the mode of administration.

Administration

The antagonist compounds of the present invention may be administered as pharmaceutical compositions either alone or in conjunction with other compounds, such as therapeutic compounds.

The pharmaceutical compositions may be administered in any effective, convenient manner including, for instance, administration by topical, oral, anal, vaginal, intravenous, intraperitoneal, intramuscular, subcutaneous, intranasal or intradermal routes among others.

The pharmaceutical compositions generally are administered in an amount effective for treatment or prophylaxis of a specific indication or indications. In general, the compositions are administered in an amount of at least about 10 μg/kg body weight. In most cases they will be administered in an amount not in excess of about 8 mg/kg body weight per day. Preferably, in most cases, dose is from about 10 μg/kg to about 1 mg/kg body weight, daily. It will be appreciated that optimum dosage will be determined by standard methods for each treatment modality and indication, taking into account the indication, its severity, route of administration, complicating conditions and the like.

The present invention is further described by the following examples. The examples are provided solely to illustrate the invention by reference to specific embodiments. These exemplification's, while illustrating certain specific aspects of the invention, do not portray the limitations or circumscribe the scope of the disclosed invention.

### EXAMPLES

All examples were carried out using standard techniques, which are well known and routine to those of skill in the art, except where otherwise described in detail. Routine molecular biology techniques of the following examples can be carried out as described in standard laboratory manuals, such as Sambrook et al., Molecular Cloning: A Laboratory Manual, 2nd Ed.; Cold Spring Harbor Laboratory Press, Cold Spring Harbor, N.Y. (1989).

### Example 1

Cloning, Expression and Purification of AFXζ

Oligonucleotides—Oligonucleotides for AFX amplification were based on the sequence in Genbank Accession number X93996. All primers were synthesized by Life Technologies (Grand Island, N.Y.). The sequences added to facilitate cloning are represented in lowercase.

| | | |
|---|---|---|
| 5-1: | 5′ GAAGACTGGCAGGAATGTGCCTCCTGG | (SEQ ID NO:3) |

3′

| | | |
|---|---|---|
| 3-1: | 5′ CGCCTGGCTCCACATCTGAAGCAGG3′ | (SEQ ID NO:4) |
| 5-2: | 5′ gacgtcgacctATGGATCCGGGGAATG | (SEQ ID NO:5) |

AG 3′

| | | |
|---|---|---|
| 3-2: | 5′ gacgtcgacTCAGGGATCTGGCTCAAA | (SEQ ID NO:6) |

G 3′

| | | |
|---|---|---|
| 5-3: | 5′ CTGTGGCAGGCTTCACTGAAC 3′ | (SEQ ID NO:7) |
| 3-3: | 5′ GCAAGTGTCAGTCGCTTCTC 3′ | (SEQ ID NO:8) |

Cloning ofAFX cDNA—AFX cDNA was amplified using primers 5-1 and 3-1 from Marathon-Ready human liver and heart cDNA libraries (Clontech, Palo Alto, Calif.). Nested primers 5-2 and 3-2 were used to perform a second round PCR. Conditions for the PCR reactions were: 94° C. for 1 minute; 94° C. for 15 seconds, 68° C. for 2 minutes, repeat for 30 cycles; 68° C. for 3 minutes and dwell at 15° C. The PCR products were digested with Sal I and cloned into the Xho I site of pcDNA6/His A (Invitrogen, Carlsbad, CA) and separately into the Sal I site of pET-30a (Novagen, Madison, Wis.). The sequences of the constructs were confirmed using dRhodamine Terminator Cycle Sequencing Kits on an ABI 377 machine (Applied Biosystems, Foster City, Calif.).

Genomic PCR and Multiple Tissue cDNA PCR of AFX—Human genomic DNA from four individuals were kindly provided by Dr. Chack Yung Yu (Columbus, Ohio). Human MTC cDNA panels I and II were purchased from Clontech. The Expand High Fidelity PCR System (Roche Molecular Biochemicals, Indianapolis, Ind.) was used to perform PCR amplifications with primers 5-3 and 3-3. PCR products were separated on 2% agarose gels. DNA was stained with Vistra Green and was visualized on the Storm Fluorlmager system (Molecular Dynamics, Sunnyvale, Calif.). The sequences of genomic PCR products were analyzed using programs in the GCG package.

Expression and Purification ofHis-tagged AFXζ—His-tagged AFXζ was expressed in an E. coli BL21 strain after induction by 0.4 mM IPTG at 30° C. for 4 hours. The bacterial fusion protein was purified using TALON metal affinity columns (Clontech) after the manufacturer's instruction.

In Vitro Translation of AFX—AFXα and AFXζ proteins were translated in vitro from pcDNA6/His-AFXα and pcDNA6/His-AFXζ templates, respectively. The TNT T7 quick coupled transcription/translation system (Promega, Madison, Wis.) was utilized according to the manufacturer's protocol. One microgram of the expression construct was used in each reaction.

### Example 2

Electrophoretic Mobility Shift Assay

The DNA binding abilities of in vitro translated AFXα, AFXζ and purified bacterial fusion protein His-tagged AFXζ were tested in a gel shift assay system (Promega). IRS elements from PEPCK, IGFBP1 and G6Pase promoters were used as probes. Briefly, oligonucleotides for sense and anti-sense strand of each IRS were annealed and double-stranded fragments were end-labeled using T4 polynucleotide kinase. The manufacturer's protocol was followed except that [γ-³³P] ATP (NEN Life Science Products, Boston, Mass.) was used in substitution of the suggested [γ-³²P] ATP to label the DNA fragment. About 2×10⁴ cpm probe was used in each binding reaction. For competition experiments, a 50 molar excess of each corresponding unlabeled oligonucleotide was added to the reaction. One hundred nanograms in vitro translated AFX protein or 200 ng purified His-tagged AFXζ was used in each binding reaction. The DNA-protein complexes were separated on 6% DNA retardation gel (Invitrogen). Gels were dried prior to autoradiography.

### Example 3

Generation of Reporter Constructs

Double-stranded DNA fragments containing each of the IRS elements from the promoters of PEPCK, IGFBP- 1 and G6Pase were cloned into the Nhe I/Bgl II site of pSEAP2-promoter vector (Clontech). Dissected fragments of the PEPCK and IGFBP-1 IRS elements, referred as PEPCK-a, PEPCK-b and IGFBP-1a, IGFBP-1b, respectively, were cloned into the same site of pSEAP2-promoter. In addition, a 501 bp fragment corresponding to the human PEPCK 5′ regulatory sequence (−458 to +43 relative to the transcription start site, abbreviated as PEPCK p) was cloned into the Bgl II/EcoR I site of the same vector.

### Example 4

Cell Culture, Transient Transfection and Reporter System Analyses

The human hepatoma cell line HepG2 was cultured in Dulbecco's Modified Eagle's Medium (DMEM) with 10% Fetal Bovine Serum (FBS, Life Technologies). Transient transfection experiments were performed in DMEM supplemented with 10% dialyzed FBS in 24-well plates. A DNA construct (0.2 μg unless otherwise specified) corresponding to IRS-SEAP, pcDNA6/His-AFXα or -AFXζ and CMV-β was added. Each set of reporter assay includes a transfection in which no AFX expression construct was added. In experiments where PKB effect was tested, 0.2 μg myr-Aktl in pUSEamp(+) (Upstate Biotechnology, Lake Placid, N.Y.), which constitutively expresses activated PKB, was included. Lipofectamine 2000 reagent (Life Technologies) was used for DNA transfection following the manufacturer's instruction. For experiment in which the cells were treated with soluble agents, the medium was changed 24 hours after the transfection. Cells were then treated with appropriate concentrations of bpV [potassium bisperoxo(1,1 0-phenanthroline) oxovanadate(V), Alexis Biochemicals, San Diego, Calif.], porcine insulin, or AICAR (5-aminoimidazole-4-carboxamide-1-β-D-ribofuranoside, Sigma, St. Louis, Mo.) in fresh media for another 15-18 hours. Secreted alkaline phosphatase (SEAP) and β-galactosidase activities were measured using Phospha-Light and Galacto-Star systems (Tropix Inc., Bedford, Mass.), respectively.

Although the present invention has been described in considerable detail with reference to certain preferred versions thereof, other versions are possible without departing from the spirit and scope of the preferred versions contained herein.

All references referred to herein are hereby incorporated by reference in their entirety.

References

1. O'Brien, R. M., and Granner, D. K. (1996) Physiol. Rev. 76, 1109-1161

2. Sasaki, K., Cripe, T. P., Koch, S. R., Andreone, T. L., Petersen, D. D., Beale, E. G., and Granner, D. K. (1984) J. Biol. Chem. 259, 15242-15251

3. Granner, D., Andreone, T., Sasaki, K., and Beale, E. (1983) Nature 305, 549-551

4. Orlowski, C. C., Ooi, G. T., Brown, D. R., Yang, Y. W., Tseng, L. Y., and Rechler, M. M. (1991) Mol. Endocrinol. 5,1180-1187

5. Powell, D. R., Suwanichkul, A., Cubbage, M. L., DePaolis, L. A., Snuggs, M. B., and Lee, P. D. (1991) J. Biol. Chem. 266, 18868-18876

6. Streeper, R. S., Svitek, C. A., Chapman, S., Greenbaum, L. E., Taub, R., and O'Brien, R. M. (1997) J. Biol. Chem. 272, 11698-11701

7. O'Brien, R. M., and Granner, D. K. (1991) Biochem. J. 278, 609-619

8. O'Brien, R. M., Lucas, P. C., Forest, C. D., Magnuson, M. A., and Granner, D. K. (1990) Science 249, 533-537

9. Suwanickul, A., Morris, S. L., and Powell, D. R. (1993) J. Biol. Chem. 268, 17063-17068

10. Goswami, R., Lacson, R., Yang, E., Sam, R., and Unterman, T. (1994) Endocrinology 134, 736-743

11. Ayala, J. E., Streeper, R. S., Desgrosellier, J. S., Durham, S. K., Suwanichkul, A., Svitek, C. A., Goldman, J. K., Barr, F. G., Powell, D. R., and O'Brien, R. M. (1999) Diabetes 48, 1885-1889

12. Kimura, K. D., Tissenbaum, H. A., Liu, Y., and Ruvkun, G. (1997) Science 277, 942-946

13. Morris, J. Z., Tissenbaum, H. A., and Ruvkun, G. (1996) Nature 382, 536-539

14. Paradis, S., and Ruvkun, G. (1998) Genes. Dev. 12, 2488-2498

15. Ogg, S., Paradis, S., Gottlieb, S., Patterson, G. I., Lee, L., Tissenbaum, H. A., and Ruvkun, G. (1997) Nature 389, 994-999

16. Lin, K., Dorman, J. B., Rodan, A., and Kenyon, C. (1997) Science. 278, 1319-1322

17. Gottlieb, S., and Ruvkun, G. (1994) Genetics 137, 107-120

18. Durham, S. K., Suwanichkul, A., Scheimann, A. O., Yee, D., Jackson, J. G., Barr, F. G., and Powell, D. R. (1999) Endocrinology 140, 3140-3146

19. Parry, P., Wei, Y., and Evans, G. (1994) Genes Chromosomes Cancer 11, 79-84

20. Corral, J., Forster, A., Thompson, S., Lampert, F., Kaneko, Y., Slater, R., Kroes, W. G., van der Schoot, C. E., Ludwig, W. D., Karpas, A., and et al. (1993) Proc. Natl. Acad. Sci. USA 90, 8538-8542

21. Medema, R. H., Kops, G. J., Bos, J. L., and Burgering, B. M. (2000) Nature 404, 782-787

22. Collado, M., Medema, R. H., Garcia-Cao, I., Dubuisson, M. L., Barradas, M., Glassford, J., Rivas, C., Burgering, B. M., Serrano, M., and Lam, E. W. (2000) J. Biol. Chem. 275, 21960-21968

23. Takaishi, H., Konishi, H., Matsuzaki, H., Ono, Y., Shirai, Y., Saito, N., Kitamura, T., Ogawa, W., Kasuga, M., Kikkawa, U., and Nishizuka, Y. (1999) Proc. Natl. Acad. Sci. USA 96, 11836-11841

24. Graff, J. R., Konicek, B. W., McNulty, A. M., Wang, Z., Houck, K., Allen, S., Paul, J. D., Hbaiu, A., Goode, R. G., Sandusky, G. E., Vessella, R. L., and Neubauer, B. L. (2000) J. Biol. Chem. 275, 24500-24505

25. Borkhardt, A., Repp, R., Haas, 0. A., Leis, T., Harbott, J., Kreuder, J., Hammermann, J., Henn, T., and Lampert, F. (1997) Oncogene 14, 195-202

26. Kops, G. J., de Ruiter, N. D., De Vries-Smits, A. M., Powell, D. R., Bos, J. L., and Burgering, B. M. (1999) Nature 398, 630-634

27. Brownawell, A. M., Kops, G. J., Macara, 1. G., and Burgering, B. M. (2001) Mol. Cell. Biol. 21, 3534-3546

28. Kops, G. J., and Burgering, B. M. (1999) J. Mol. Med. 77, 656-665

29. Tomizawa, M., Kumar, A., Perrot, V., Nakae, J., Accili, D., Rechler, M. M., and Kumaro, A. (2000) J. Biol. Chem. 275, 7289-7295

30. Winder, W. W., and Hardie, D. G. (1999) Am. J. Physiol. 277, E1-10

31. Furuyama, T., Nakazawa, T., Nakano, I., and Mori, N. (2000) Biochem. J. 349, 629-634

32. Imai, E., Stromstedt, P. E., Quinn, P. G., Carlstedt-Duke, J., Gustafsson, J. A., and Granner, D. K. (1990) Mol. Cell. Biol. 10, 4712-4719

33. Mitchell, J., Noisin, E., Hall, R., O'Brien, R., Imai, E., and Granner, D. (1994) Mol. Endocrinol. 8, 585-594

34. O'Brien, R. M., Noisin, E. L., Suwanichkul, A., Yamasaki, T., Lucas, P. C., Wang, J. C., Powell, D. R., and Granner, D. K. (1995) Mol. Cell. Biol. 15, 1747-1758

35. Unterman, T. G., Fareeduddin, A., Harris, M. A., Goswami, R. G., Porcella, A., Costa, R. H., and Lacson, R. G. (1994) Biochem. Biophys. Res. Commun. 203, 1835-1841

36. Guo, S., Rena, G., Cichy, S., He, X., Cohen, P., and Unterman, T. (1999) J. Biol. Chem. 274, 17184-17192

37. Lochhead, P. A., Salt, I. P., Walker, K. S., Hardie, D. G., and Sutherland, C. (2000) Diabetes 49, 896-903

38. Weigelt, J., Climent, I., Dahlman-Wright, K., and Wikstrom, M. (2001) Biochemistry 40, 5861-5869

39. Hardie, D. G., and Carling, D. (1997) Eur. J. Biochem. 246, 259-273

40. Corton, J. M., Gillespie, J. G., Hawley, S. A., and Hardie, D.G. (1995) Eur. J. Biochem. 229, 558-565

What is claimed is:

1. An isolated polynucleotide comprising a member selected from the group consisting of:
(a) a polynucleotide having at least 90% identity to a polynucleotide encoding a polypeptide comprising an amino acid sequence as set forth in SEQ ID NO:2; and
(b) a polynucleotide which is complementary to the polynucleotide of (a).

2. The polynucleotide of claim 1 wherein the polynucleotide is DNA.

3. The polynucleotide of claim 2 which encodes the polypeptide comprising an amino acid sequence as set forth in SEQ ID NO:2.

4. The polynucleotide of claim 1 wherein the polynucleotide is RNA.

5. The polynucleotide of claim 1 comprising the sequence as set forth in SEQ ID NO:1.

6. An isolated polypeptide comprising an amino acid sequence which is at least 90% identical to the amino acid sequence set forth in SEQ ID NO:2.

7. The polypeptide of claim 6 comprising the amino acid sequence of SEQ ID NO:2.

8. A process for producing a human AFXζ polypeptide comprising:
(a) transforming a host cell with a vector comprising DNA which upon expression encodes the polypeptide of claim 6;
(b) culturing the host cell under conditions promoting expression of the polypeptide; and
(c) recovering the expressed polypeptide.

9. A human AFXζ polypeptide prepared by the process of claim 8.

10. A process for producing a cell which expresses a polypeptide comprising transforming the cell with a vector comprising DNA which upon expression encodes the polypeptide of claim 6.

11. A cell prepared by the process of claim 10.

12. A host cell transformed with a vector comprising DNA which upon expression encodes the polypeptide of claim 6.

13. An antibody which binds to the polypeptide of claim 6.

14. A method of producing an antibody comprising: purifying the antibody produced by the mammal; and recovering the antibody.

15. A method for identifying substances that antagonize or prevent the activity of AFXζ protein comprising:
(a) contacting AFXζ protein with a substance; and
(b) determining the ability of the substance to antagonize or prevent the activity of AFXζ protein as compared to a control.

16. A substance identified by the method of claim 15.

17. A method for treating a disease associated with AFXζ protein comprising administering a therapeutically effective amount of the substance of claim 16 to a patient in need thereof.

18. A method for preventing or reducing hepatic gluconeogenesis comprising administering a therapeutically effective amount of the substance of claim 16 to a patient in need thereof.

19. A method for treating diabetes comprising administering a therapeutically effective amount of the substance of claim 16 to a,patient in need thereof.

20. A pharmaceutical composition comprising the substance of claim 16 and a pharmaceutically acceptable carrier.

21. A method for identifying compounds which agonize or antagonize AFXζ-regulated genes comprising:
a) preparing an AFXζ expression construct and an IRS-containing reporter construct;
b) introducing the constructs into a eukaryotic cell;
c) exposing the cells to a candidate compound; and
d) determining if the compounds augment or reduce the AFXζ dependent gene activation.

22. A compound identified by the method of claim 21.

23. A method for treating a disease comprising administering a therapeutically effective amount of the compound of claim 22 to a patient in need thereof.

24. The method of claim 23 wherein the disease is diabetes, atherosclerosis or dyslipidemia.

25. A pharmaceutical composition comprising the compound of claim 22 and a pharmaceutically acceptable carrier.

26. A method for identifying genes which agonize or antagonize AFXζ-regulated genes comprising:
a) preparing an AFXζ expression construct and an IRS-containing reporter construct;
b) introducing the constructs into a eukaryotic cell;
c) introducing an expression construct containing a candidate gene into the cell; and
d) determining if the gene augments or reduces the AFXζ dependent gene activation.

27. A gene identified by the method of claim 26.


SEQUENCE LISTING (8 sequences)

SEQ ID NO: 1 - DNA, 1353 bp, Homo Sapien
atggatccggggaatgagaattcagccacagaggccgccgcgatcatagacctagatcccgacttcgaaccccagagccgtccccgctcctgcacctggccccttccccgaccagagatcgctaaccagccgtccgagccgcccgaggtggagccagatctgggggaaaaggccattgaaagcgccccggagaagcgactgacacttgcccagatttacgagtggatggtccgtactgtaccctacttcaaggacaagggtgacagcaacagctcagcaggatggaagaactcgatccgccacaacctgtccctgcacagcaagttcatcaaggttcacaacgaggccaccggcaaaagctcttggtggatgctgaaccctgagggaggcaagagcggcaaagccccccgccgccgggccgcctccatggatagcagcagcaagctgctccggggccgcagtaaagcccccaagaagaaaccatctgtgctgccagctccacccgaaggtgccactccaacgagccctgtcggccactttgccaagtggtcaggcagcccttgctctcgaaaccgtgaagaagccgatatgtggaccaccttccgtccacgaagcagttcaaatgccagcagtgtcagcacccggctgtcccccttgaggccagagtctgaggtgctggcggaggaaataccagcttcagtcagcagttatgcagggggtgtccctcccaccctcaatgaaggtctagagctgttagatgggctcaatctcacctcttcccattccctgctatctcggagtggtctctctggcttctctttgcagcatcctggggttaccggccccttacacacctacagcagctcccttttcagcccagcagaggggcccctgtcagcaggagaagggtgcttctccagctcccaggctctggaggccctgctcacctctgatacgccaccaccccctgctgacgtcctcatgacccaggtagatcccattctgtcccaggctccgactcttctgttgctgggggggcttccttcctccagtaagctggccacgggcgtcggcctgtgtcccaagcccctagaggctcgaggccccagcagtctggttcccaccctttctatgatagcaccacctccagtcatggcaagtgcccccatccccaaggctctggggactcctgtgctcacaccccctactgaagctgcaagccaagacagaatgcctcaggatctagatcttgatatgtatatggagaacctggagtgtgacatggataacatcatcagtgacctcatggatgagggcgagggactggacttcaactttgagccagatccctga

SEQ ID NO: 2 - protein, 505 aa, Homo Sapien
MDPGNENSATEAAAIIDLDPDFEPQSRPRSCTWPLPRPEIANQPSEPPEVEPDLGEKVHTEGRSEPILLPSRLSEPAGGPQPGILGAVTGPRKGGSRRNAWGNQSYAEFISQAIESAPEKRLTLAQIYEWMVRTVPYFKDKGDSNSSAGWKNSIRHNLSLHSKFIKVHNEATGKSSWWMLNPEGGKSGKAPRRRAASMDSSSKLLRGRSKAPKKKPSVLPAPPEGATPTSPVGHFAKWSGSPCSRNREEADMWTTFRPRSSSNASSVSTRLSPLRPESEVLAEEIPASVSSYAGGVPPTLNEGLELLDGLNLTSSHSLLSRSGLSGFSLQHPGVTGPLHTYSSSLFSPAEGPLSAGEGCFSSSQALEALLTSDTPPPPADVLMTQVDPILSQAPTLLLLGGLPSSSKLATGVGLCPKPLEARGPSSLVPTLSMIAPPPVMASAPIPKALGTPVLTPPTEAASQDRMPQDLDLDMYMENLECDMDNIISDLMDEGEGLDFNFEPDP

SEQ ID NO: 3 - DNA, 27 bp, Artificial Sequence
  Oligonucleotide Primer
gaagactggcaggaatgtgcctcctgg

SEQ ID NO: 4 - DNA, 25 bp, Artificial Sequence
  Oligonucleotide Primer
cgcctggctccacatctgaagcagg

SEQ ID NO: 5 - DNA, 29 bp, Artificial Sequence
  Oligonucleotide Primer
gacgtcgacctatggatccggggaatgag

SEQ ID NO: 6 - DNA, 28 bp, Artificial Sequence
  Oligonucleotide Primer
gacgtcgactcagggatctggctcaaag

SEQ ID NO: 7 - DNA, 21 bp, Artificial Sequence
  Oligonucleotide Primer
ctgtggcaggcttcactgaac

SEQ ID NO: 8 - DNA, 20 bp, Artificial Sequence
  Oligonucleotide Primer
gcaagtgtcagtcgcttctc